(19) United States
(12) Patent Application Publication
(10) Pub. No.: US 2025/0208131 A1
(43) Pub. Date: Jun. 26, 2025

(54) Diagnosis of Congenital Heart Block

(71) Applicant: The Hospital for Sick Children, Toronto (CA)

(72) Inventor: Robert Hamilton, Toronto (CA)

(21) Appl. No.: 18/845,563

(22) PCT Filed: Mar. 10, 2023
(86) PCT No.: PCT/CA2023/050319

§ 371 Date: Sep. 10, 2024

Related U.S. Application Data
(60) Provisional application No. 63/318,450, filed on Mar. 10, 2022.

(51) Int. Cl. G01N 33/564; G01N 33/68

(52) U.S. Cl. CPC G01N 33/564; G01N 33/6872; G01N 2333/705; G01N 2333/90; G01N 2800/32; G01N 2800/38

(57) ABSTRACT

A method of diagnosing congenital heart block or risk of congenital heart block in a fetal or infant subject is provided. The method includes the step of detecting in a biological sample obtained from the mother of the subject one or more maternal autoantibodies which bind to target cardiomyocyte proteins of the subject. A kit for use in to detect such maternal autoantibodies is also provided.

### FIELD OF THE INVENTION

The present invention generally relates to congenital heart block and methods of diagnosing congenital heart block in a fetus or infant.

### BACKGROUND

Autoimmune congenital heart block (CHB) is a condition that affects the fetus of a woman with (or at risk for) autoimmune systemic lupus erythematosus (SLE) or Sjögren's syndrome, and/or a woman who has had a previous child with CHB. The typical anti-Ro antibodies of these conditions are usually present in pregnancies when the fetus or newborn is identified with CHB, but only 2% of anti-Ro positive pregnancies result in CHB. Thus, it is difficult to target potential preventive therapies (e.g. hydroxychloroquine) to pregnancies truly at risk for CHB. Moreover, anti-Ro positive pregnancies must be screened frequently during early second trimester by fetal echocardiography to identify the onset of CHB to provide secondary therapies (e.g. fluorinated steroids or chronotropic agents). Substantial mortality and morbidity persist in pregnancies affected by CHB.

Accordingly, it would be desirable to develop more accurate methods of identifying pregnancies at risk for CHB.

### SUMMARY

Novel maternal autoantibodies that cross-react with fetal heart conduction system proteins have now been identified that discriminate pregnancies affected by congenital heart block from those not affected by congenital heart block.

Accordingly, in one aspect, a method of diagnosing congenital heart block or risk of congenital heart block in a fetal or infant subject is provided. The method comprises the steps of: i) contacting a biological sample obtained from the mother of the subject with one or more target cardiomyocyte proteins; ii) detecting one or more maternal autoantibodies from the sample which bind with at least one of the target cardiomyocyte proteins; and iii) diagnosing the subject with congenital heart block or risk of congenital heart block when the sample contains one or more maternal autoantibodies that bind with at least one of the target cardiomyocyte proteins.

In another aspect of the invention, a method comprising the steps of: i) contacting a biological sample obtained from the mother of a fetal or infant subject with one or more fetal or infant cardiomyocyte proteins found in the ventricular myocardium; and ii) detecting one or more maternal autoantibodies from the biological sample which bind to at least one of the cardiomyocyte proteins, is provided.

In a further aspect, a kit useful for the diagnosis of CHB is provided. The kit comprises AT1A1, MYBPC3, HSPA5 and annexin1 antigens, and optionally one or more antigens selected from AT2A2, CO1A2 and vimentin, immobilized on a solid support.

These and other embodiments of the invention are described by reference to the following figures.

### BRIEF DESCRIPTION OF THE FIGURES

FIG. 1: A)-C) Dot blots illustrating that serum from three anti-Ro/La mothers who delivered normal newborns contain only anti-52 kDa and anti-60 kDa Ro and anti-La antibodies. D)-F) Dot blots illustrating that serum from three mothers of newborns with CHB demonstrate at least 30 anti-fetal cardiac antibodies when measured at the time of delivery.

FIG. 2: Dot blots identifying antibodies (Abs) in CHB+ maternal sera, four specific Abs are found in early gestation (15.9 to 18.7 weeks) before CHB (top 4 panels), compared to after CHB has been established later in pregnancy (19.7 to 34 weeks gestation, bottom 3 panels), using proteins from stem cell-derived atrioventricular node pacemaker-like cells (AVNPLC).

FIG. 3. Multi-lane western blots demonstrating antibodies to 7 protein targets in sera from pregnancies with established CHB (panel B) versus sera from Ro+ CHB− (panel C; p<0.015 by patient, p<0.005 by serum) and female young adult control sera (panel D; p<0.015 by patient, p<0.005 by serum). The 4 samples in panels A1 and A2 were drawn prior to the onset of CHB and represent that a subset of four antibodies can be predictive for subsequent CHB (p<0.0286 by patient).

FIG. 4. Single nuclear transcriptome of a fetal AV junction identifies AV nodal cells (AVN, left) that express the protein target (dashed circle, middle). Ro 52 is essentially not expressed in the AV junction, with 60 kDa Ro having only moderate expression, and not specific to AVN (right).

FIG. 5. An illustration of the human AT1A1 protein.

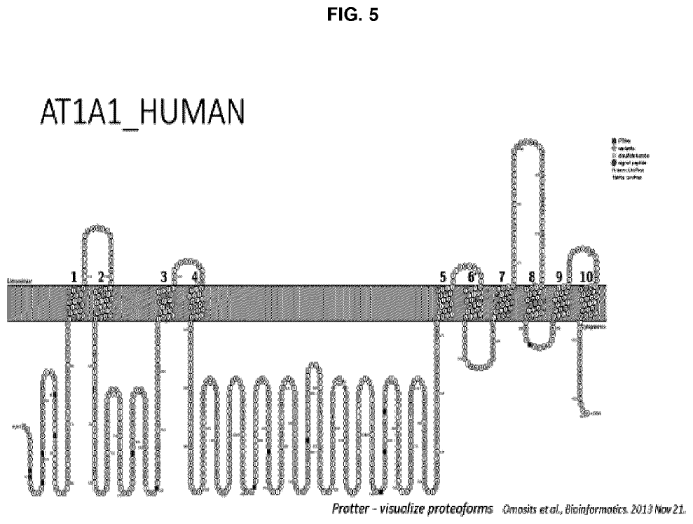

FIG. 6. The amino acid sequence of human AT1A1 protein.

FIG. 7. The amino acid sequence of human MYBPC3 protein.

FIG. 8. The amino acid sequence of human AT2A2 protein.

FIG. 9. The amino acid sequence of human CO1A2 protein.

FIG. 10. The amino acid sequence of human HSPA5 protein.

FIG. 11. The amino acid sequence of human annexin1 protein.

FIG. 12. The amino acid sequence of human vimentin protein.

FIG. 13. Pixel Counts from Western Blots of proteins, A) ATP1A1; B) Annexin 1; C) HSPA5 protein; D) MYBPC3; E) Vim; F) ATP2A2; and G) COLIA, in relation to time point of CHB diagnosis, and controls. Populations of patients sampled includes two control groups: normal young women) and Ro+ mothers with CHB− offspring). Three time points of CHB diagnosis are also displayed: Pre CHB+ pregnancy, CHB+ pregnancy, and CHB+ offspring with samples collected during the post natal period. All data points are displayed along with means and standard deviations.

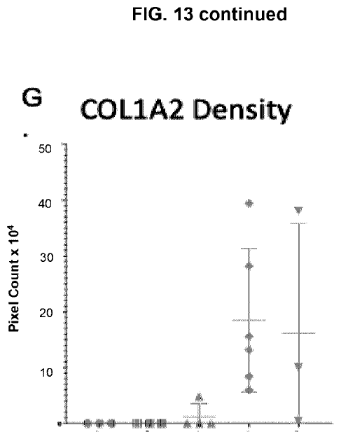

FIG. 14. A workflow and comparison chart of a Discovery and Validation cohorts.

FIG. 15. AT1A1 peptide map illustrating antigenic regions.

### DETAILED DESCRIPTION

A method of diagnosing congenital heart block (CHB) or risk of CHB in a mammalian fetal or infant subject is provided. The method comprises the step of contacting a biological sample obtained from the mother of the subject with one or more target cardiomyocyte proteins; detecting one or more maternal autoantibodies from the sample which bind with at least one of the target cardiomyocyte proteins; and diagnosing the subject with congenital heart block or risk of congenital heart block when the sample contains one or more maternal autoantibodies that bind with at least one of the target cardiomyocyte proteins.

The present method is useful to diagnose CHB, or risk of CHB, in a subject which is a fetus or an infant. The method is useful to detect CHB or risk of CHB from conception to birth. Preferably, the method may be used to detect CHB, or risk thereof, in a fetus from 12 weeks to birth, or in an infant from newborn to 12 months.

The method comprises the step of detecting in a biological sample obtained from the mother of the subject, one or more maternal antibodies that react with a target cardiomyocyte protein of the fetus or newborn subject. The sample may be obtained from the mother from conception to birth and postnatally. For example, maternal samples may be obtained from the mother when the fetus is about 4 weeks, 5 weeks, 6 weeks, 7, weeks, 8 weeks, 10 weeks, 12 weeks, 13 weeks, 14 weeks, 15 weeks, 16 weeks, 17 weeks, 18 weeks, 19 weeks, 20 weeks, 25 weeks, 30 weeks or 40 weeks old, or more up to birth. Maternal samples may also be obtained postnatally, for example, immediately following birth, or 1 week, 4 weeks, 8 weeks, 12 weeks, 16 weeks, 20 weeks, or 6-12 months postnatally.

The maternal biological sample may be blood, serum, plasma, urine, saliva, cerebrospinal fluid, amniotic fluid or other bodily fluids. Maternal tissues that express autoantibodies may also be used. The biological sample may be obtained using methods well-established in the art, and may be obtained directly from the mammal, or may be obtained from a sample previously acquired from the mammal which has been appropriately stored for future use (e.g. stored at 4° C.). Samples may require processing prior to use in the present method. For example, the sample may be purified using methods such as filtration and/or centrifugation to remove cellular and other debris therefrom prior to use. For tissue samples, autoantibody extract may be obtained for use in the method. An amount of sample of at least about 100 μl, e.g. 100 μl of diluted human serum (1:100 dilution in blocking buffer) may be used to conduct the present method. The term “mammal” is used herein to refer to both human and non-human mammals including, but not limited to, cats, dogs, horses, cattle, goats, sheep, pigs and the like.

Once a suitable biological sample is obtained, it is determined whether or not the sample contains autoantibodies that target one or more cardiomyocyte proteins of the subject. The term “cardiomyocyte” refers to proteins which are produced by cardiac muscle cells. Autoantibodies against one or more cardiomyocyte proteins, preferably proteins in ventricular myocardium, including AV-node-like pacemaker cells, are detected by contacting the sample with one or more of the target cardiomyocyte protein antigens in either full-length form or as a fragment of the full-length protein which comprises an antibody-binding epitope, i.e. an antigenic fragment. In one embodiment, the cardiomyocyte proteins include one or more of AT1A1, MYBPC3, AT2A2, CO1A2, HSPA5, ANXA1 (annexin1) and vimentin protein. The sample is contacted with the proteins/fragments under conditions which permit binding of antibody to the protein/fragment.

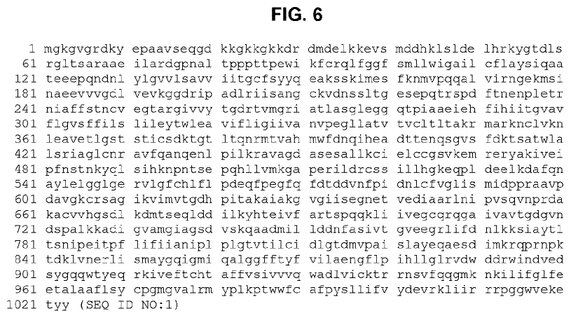

AT1A1 refers to the sodium/potassium-transporting ATPase subunit alpha-1 cardiomyocyte protein, encoded by the gene, ATP1A1. The term, AT1A1 encompasses herein mammalian AT1A1, including functionally equivalent isoforms thereof and orthologs. In one embodiment, the AT1A1 is human AT1A1, which may have a sequence as depicted by NCBI Reference Sequence: NP_000692.2 (isoform a as shown in FIG. 6), or a related isoform sequence such as depicted by NCBI ref. sequences: NP_001153705.1, NP_001153706.1 or XP_016856849.1.

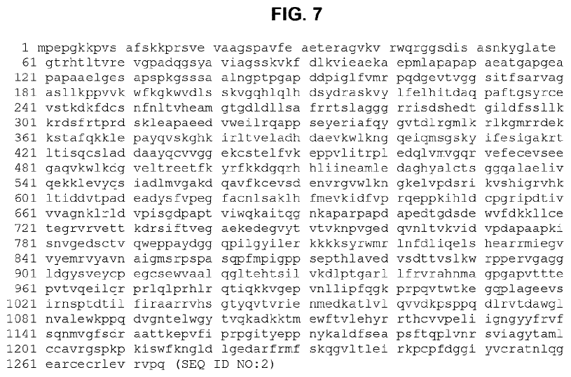

MYBPC3 refers to myosin-binding protein C, cardiac-type, or myosin binding protein C3, and is encoded by the MYBPC3 gene. The term, MYBPC3 encompasses herein mammalian MYBPC3, including functionally equivalent isoforms thereof and orthologs. In one embodiment, the MYBPC3 is human MYBPC3, which may have a sequence as depicted by NCBI Reference Sequence: NP_000247 (as shown in FIG. 7), or NP_000247.2, or a related isoform sequence.

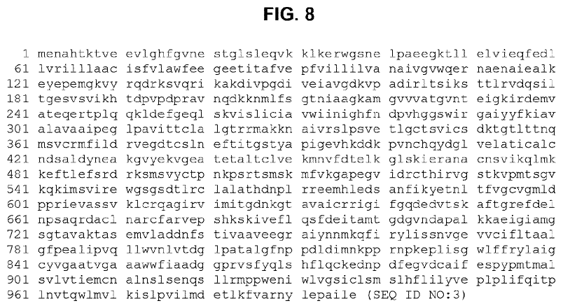

AT2A2 refers to sarcoplasmic/endoplasmic reticulum calcium ATPase 2(SERCA2) encoded by the gene, ATP2A2. The term, AT2A2 encompasses herein mammalian AT2A2, including functionally equivalent isoforms thereof and orthologs. In one embodiment, the AT2A2 is human AT2A2, which may have a sequence as depicted by NCBI Reference Sequence: NP_001672.1 (isoform a as shown in FIG. 8), or an isoform sequence such as that of isoform b depicted by NCBI ref. sequences: NP_733765.1.

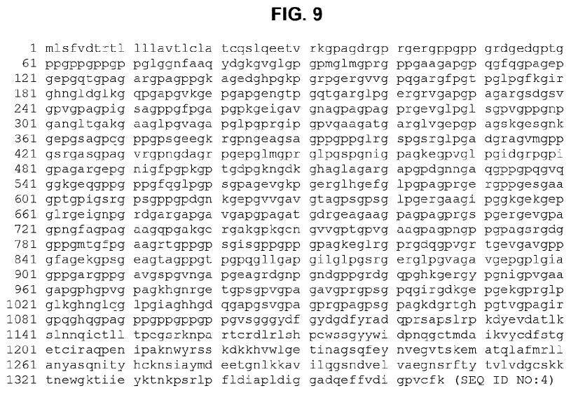

CO1A2 refers to Collagen Type I Alpha 2 Chain protein encoded by the gene, COLIA2. The term, CO1A2 encompasses herein mammalian CO1A2, including functionally equivalent isoforms thereof and orthologs. In one embodiment, the CO1A2 is human CO1A2, which may have a sequence as depicted by NCBI Reference Sequence: NP_000080 (as shown in FIG. 9), or a related isoform sequence.

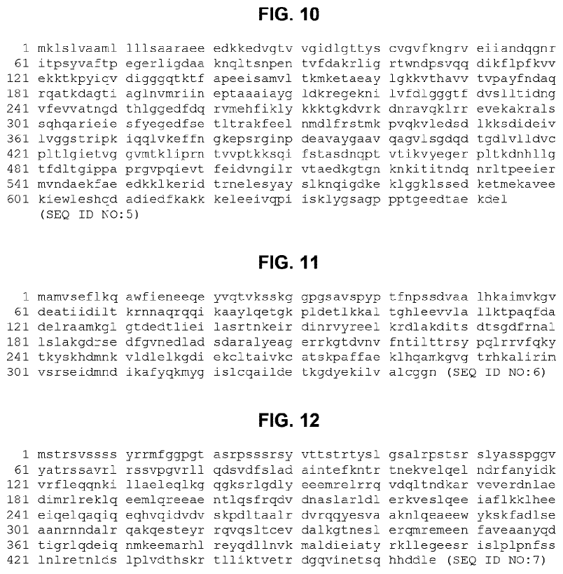

HSPA5 refers to endoplasmic reticulum chaperone binding immunoglobulin protein (BiP), also known as (GRP-78), heat shock 70 kDa protein 5 (HSPA5) or (Byun1), which is encoded by the HSPA5 gene. The term, HSPA5, encompasses herein mammalian HSPA5, including functionally equivalent isoforms thereof and orthologs. In one embodiment, the HSPA5 is human HSPA5, which may have a sequence as depicted by NCBI Reference Sequence: NP_005338.1 (as shown in FIG. 10) or a related isoform sequence.

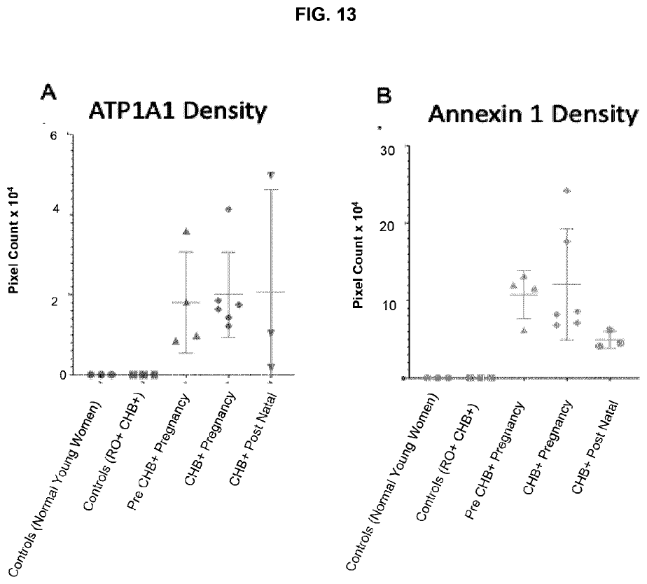

Annexin1, also known as lipocortin I, is a protein that is encoded by the ANXA1 gene in humans. The term, annexin1, encompasses herein mammalian annexin1, including functionally equivalent isoforms thereof and orthologs. In one embodiment, the annexin1 is human annexin1, which may have a sequence as depicted by NCBI Reference Sequence: NP_000691.1 (as shown in FIG. 11), or an isoform sequence such as that depicted by NCBI Sequences: XP_016870146.1 (isoform X1) or XP_011516911.1 (isoform X2).

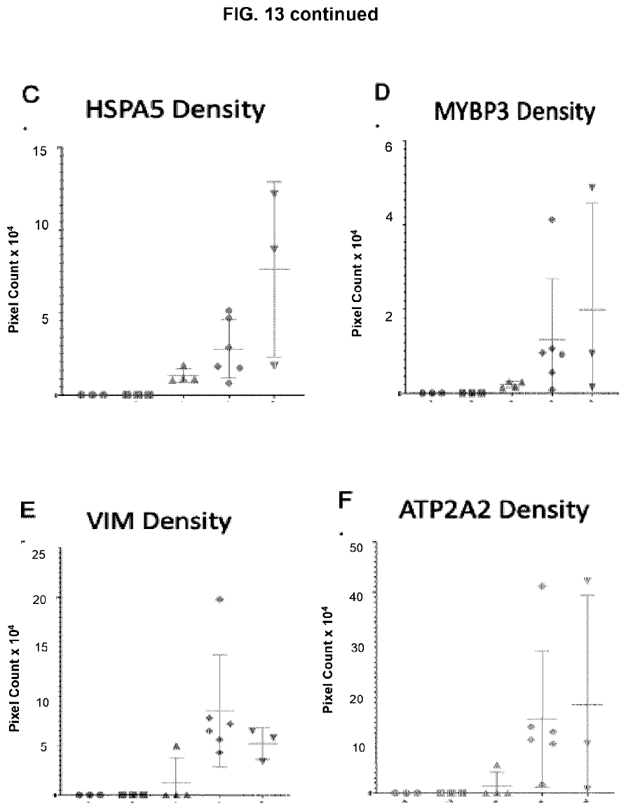

Vimentin is a structural type III intermediate filament (IF) protein encoded by the VIM gene. The term, vimentin, encompasses herein mammalian vimentin, including functionally equivalent isoforms thereof and orthologs. In one embodiment, the vimentin is human vimentin, which may have a sequence as depicted by NCBI Reference Sequence: NP_003371.2 (as shown in FIG. 12), or an isoform sequence such as that depicted by NCBI Sequence: XP_006717563.1 (isoform X1).

Methods for detecting autoantibodies in a sample in accordance with aspects of the invention are generally known in the art, and utilize antibody-specific antigens which bind to the autoantibody, capturing the antibody for detection. Such methods include, but are not limited to, immunoprecipitation assays in which antibody-antigen (Ab-Ag) complex aggregates are detected; immunoblotting whereby Ab-Ag aggregates are trapped on membranes and then detected with a secondary antibody (e.g. Western blotting); and immunosorbent assays, which utilize a tagged secondary antibody and permit quantification of the primary target autoantibody (e.g. enzyme-linked immunoassay (ELISA), latex agglutination, or microparticle enzyme immunosorbent assay (MEIA)).

Antigens suitable to detect maternal autoantibodies to fetal or infant cardiomyocyte proteins may include full-length protein, such as full-length cardiomycocyte proteins such as AT1A1, MYBPC3, AT2A2, CO1A2, HSPA5, annexin1 or vimentin. Alternatively, the antigen may be an antigenic fragment of a full-length cardiomyocyte protein. Antigenic fragments may be derived from extracellular or intracellular regions of the protein, such as N-terminal or C-terminal regions, extracellular or intracellular internal regions of the protein or transmembrane portions of the protein. Suitable antigens may be based on the protein sequences of the cardiomyocyte protein(s), such as AT1A1, MYBPC3, AT2A2, CO1A2, HSPA5, annexin1 and vimentin protein sequences provided herein, or available in public databases, including antigenic fragments derived from these sequences. For example, antigenic fragments of ATA1 may be derived from intracellular regions, such as those spanning amino acid residues 1-95, 150-293, 340-775 and 938-965 or other regions. Antigenic fragments specific for a maternal autoantibody of a cardiomyocyte protein comprises at least about 10-50 amino acids from an antigenic region of the cardiomyocyte protein. Antigenic fragments of any of MYBPC3, AT2A2, CO1A2, HSPA5, annexin1 or vimentin may be similarly derived. Exemplary antigenic peptides derived from AT1A1 include peptides comprising amino acid residues 1-20, 151-170, 196-215, 256-275, 376-395, 421-440, 451-470, 511-530, 601-615 and/or residues 951-965 of AT1A1.

Depending on the method of detection utilized, the antigen (protein or fragment thereof) may be bound to or immobilized on a solid support, such as a nitrocellulose, polyvinylidene difluoride (PVDF), or cationic nylon membranes, or microparticles such as latex microbeads. As known to those of skill in the art, in order to prevent nonspecific binding on the solid support, free binding sites on the support are blocked using a suitable blocking buffer, e.g. milk, normal serum or purified proteins. Conditions suitable for binding antigen to the selected solid support are used, for example, incubation at an appropriate temperature in a suitable buffer. Following binding, the solid support is washed to remove unbound and/or non-specifically bound materials. A physiological buffer such as Tris buffered saline (TBS) or phosphate buffered saline (PBS) may be used, optionally including additives such as a detergent (e.g. 0.05% Tween™ 20). The solid support may comprise a single antigen, or multiple antigens, either from the same cardiomyocyte protein or from different cardiomyocyte proteins. For example, in one embodiment, a solid support is prepared comprising a single antigen, e.g. an antigenic fragment of AT1A1. In another embodiment, a solid support is prepared comprising multiple antigens of AT1A1, e.g. full-length AT1A1, or multiple antigenic fragments of AT1A1. In further embodiments, a solid support is prepared comprising multiple cardiomyocyte proteins or multiple antigenic fragments from two or more of a cardiomyocyte protein, e.g. AT1A1, MYBPC3, AT2A2, CO1A2, HSPA5, annexin1 and vimentin protein.

The sample is then combined with one or more antigens to permit binding of autoantibodies in the sample to target antigen. Autoantibody bound to the target antigen can then be detected. In one embodiment, antigen-bound autoantibody is detected using a secondary antibody that will bind to the autoantibody and which is detectable. If the sample is a human sample, then an anti-human secondary antibody may be used, for example, anti-human antibody derived from a non-human mammal (e.g. goat, rabbit, mouse, rat, chicken, pig, cow, sheep, donkey) against human immunoglobulin such as immunoglobulin G (lgG). If the sample is a non-human sample, then a suitable secondary antibody may be used to detect the target autoantibody which may be obtained from a different non-human mammal. To detect antigen-bound target autoantibody, the sample is contacted with the secondary antibody under conditions suitable for binding and then washed to remove unbound reagent, e.g. secondary antibody. The secondary antibody is labelled with any suitable detectable label, either prior or subsequent to target autoantibody binding using established protocols. Suitable labels include, but are not limited to, an enzyme label such as glucose oxidase, horseradish peroxidase (HRP) or alkaline phosphatase (AP); a fluorescent label such as ethidium bromide, fluorescein, rhodamine, phycoerythrin, cyanine, coumarin, green fluorescent protein and derivatives thereof; an affinity label such as biotin/streptavidin labelling; or radioactive labels.

The presence of target autoantibody is detected by detecting the presence of the selected label using methods known to those of skill in the art. For example, to detect enzyme labels, an appropriate enzyme substrate is added to the sample and enzyme activity is detected by chromogenic, chemiluminescent or fluorescent outputs. Examples of commonly used substrates for HRP include chromogenic substrates, 3,3′,5,5′-tetramethylbenzidine, 3,3′-diaminobenzidine and 2,2′-azino-bis(3-ethylbenzothiazoline-6-sulphonic acid), and chemiluminescent substrates such as luminol. Examples of commonly used substrates for AP include chromogenic substrates, 4-nitrophenyl phosphate and 4-methylumbelliferyl phosphate. Biotin-streptavidin binding may be similarly detected.

Microparticle enzyme immunosorbent assay (MEIA) may be utilized to detect the target autoantibodies in accordance with the present method. MEIA is a technique that utilizes very small microparticles in liquid suspension as a solid-phase support. Specific reagent antibodies are covalently bound to the microparticles. Antigen (e.g. a cardiomyocyte protein or antigenic fragment(s) thereof) is then bound to the immobilized antibody. Sample is added to the microparticles, and target autoantibody, if present, binds to the antigen. Binding of the autoantibody is detected using an enzyme-based detection reaction in which enzyme is bound to the autoantibody and fluorescence is detected on addition of enzyme substrate to the reaction microparticle mix.

Latex agglutination may also be used in which latex particles are coated with antigen. Sample is added to the latex particles. If target autoantibody is present, it will bind with the antigen resulting in agglutination of the latex particles.

Methods which permit detection of antibodies directly in solution may also be used. For example, an antibody sensor platform, known as LUMinescent AntiBody Sensor (LUMABS) based on bioluminescence resonance energy transfer (BRET) may be used to detect target autoantibody in the maternal sample. LUMABS are single-protein sensors that consist of the blue-light emitting luciferase, NanoLuc, connected via a semiflexible linker to the green fluorescent acceptor protein mNeonGreen, which are kept close together using helper domains. Binding of an antibody to antigen sequences flanking the linker disrupts the interaction between the helper domains, resulting in a large decrease in BRET efficiency. The resulting change in color of the emitted light from green-blue to blue can be detected directly in blood plasma, even at picomolar concentrations of antibody.

In one embodiment, the presence of an autoantibody to AT1A1 in the maternal sample is detected, and is indicative of CHB or risk of CHB. In this regard, AT1A1 autoantibody is detected in a maternal sample obtained from conception to birth, or postnatally. In an embodiment, the sample is obtained when the fetus is at least about 12 to 18 weeks old. In another embodiment, AT1A1 autoantibody is detected either prior to onset of CHB in the fetus or subsequent to onset of CHB in the fetus.

In another embodiment, the presence of one or more autoantibodies to a cardiomyocyte protein selected from AT1A1, MYBPC3, HSPA5 and Annexin1 is indicative of risk of CHB. In this regard, the autoantibody is detected in a maternal sample obtained from conception to birth, or postnatally. In an embodiment, the maternal sample is obtained when the fetus is at least about 12 to 18 weeks old. Alternatively, the presence of one or more of these autoantibodies in the maternal sample is indicative of CHB. The method may comprise detection of each of AT1A1, MYBPC3, HSPA5 and annexin1 in a maternal sample pre- or post-natally.

In another embodiment, the presence of one or more of autoantibodies in the maternal sample to a cardiomyocyte protein selected from AT1A1, MYBPC3, AT2A2, CO1A2, HSPA5, annexin1 and vimentin is indicative of CHB, or risk thereof. The method may comprise the detection of autoantibodies to each of AT1A1, MYBPC3, AT2A2, CO1A2, HSPA5, annexin1 and vimentin in the maternal sample obtained either pre- or post-natally.

Severity of the CHB condition may also be diagnosed based on the autoantibody detection results. For example, the greater the level of autoantibody, and/or the greater the number of different autoantibodies detected in the sample, the more severe the CHB. Severity of CHB may also be determined based on ventricular escape rate.

Following diagnosis of CHB, or risk of CHB, preventative or secondary therapies may be administered to the subject to potentially improve the prognosis. The recommended treatment will depend on the nature of the CHB and its severity. Preventive therapies to treat a subject at risk of CHB prior to birth may include administration to the mother of hydroxychloroquine. At-risk pregnancies are also generally screened frequently during early second trimester by fetal echocardiography to identify the onset of CHB in order to initiate secondary therapies (such as fluorinated steroids, or chronotropic agents administered to the mother). For treatments in subjects determined to have CHB prior to birth, medication may be administered to the mother to decrease inflammation, including adrenocorticosteroids such as dexamethasone, or administration of sympathomimetics such as α-adrenergic agonists, β-adrenergic agonists, and dopaminergic agonists or stimulants, examples of which include, but are not limited to, dobutamine, dopamine, phenylephrine, norepinephrine, epinephrine, and isoproterenol. Following birth, CHB is monitored. For more severe CHB, the subject may require a pacemaker.

The present method advantageously provides a means to diagnose CHB or risk of CHB in a fetus or infant with enhanced sensitivity in comparison to prior methods based on maternal anti-Ro antibodies. The current method more specifically targets the subject, i.e. fetus or newborn, by detecting maternal antibodies that target cardiomyocyte proteins of the subject.

In another aspect of the invention, a kit is provided comprising a panel of cardiomyocyte antigens useful for the diagnosis of CHB. The antigens, including full-length protein or antigenic fragment thereof, are immobilized on a solid support and may be used in a method of diagnosis as described herein. The antigens comprise AT1A1, MYBPC3, HSPA5 and annexin1, and may optionally include one or more antigens selected from AT2A2, CO1A2 and vimentin. The kit may additionally include reagents useful to conduct the method.

Embodiments of the invention are described by reference to the following specific examples which are not to be construed as limiting.

### Example 1

A study was conducted to determine maternal autoantibodies that target proteins isolated from mid-second trimester fetal heart tissue.

Materials: The study relied on banked sera from several studies of pregnancies to women considered at risk to have a fetus for CHB, and who were known to have anti-Ro antibodies. These included pregnancies to women with identified SLE or Sjögren's syndrome, as well as pregnancies to women who had a prior child with CHB. The initial studies evaluated serum antibodies from late in pregnancies where CHB had already been established in the fetus and assessed using 2D gels of proteins solubilized from an early second trimester fetal heart to identify targets of these autoantibodies.

A second set of studies was performed using sequential sera throughout pregnancies affected by CHB, including four where serum was sampled prior to CHB onset, and these were assessed using 2D gels of proteins solubilized from stem cell-derived atrioventricular node pacemaker-like cells AVNPLCs. Sera were also assessed by western blot against commercial proteins corresponding to protein identified by mass spectrometry of reactive regions (spots) from the 2D gels. Finally, these techniques were used to assess maternal sera from the four pregnancies that were drawn prior to the onset of CHB in the fetus. For all studies, sera from anti-Ro positive pregnancies that resulted in normal offspring were used as controls, as well as sera from normal young women.

Normal fetal myocardium from 18 to 22-week elective terminations were obtained from Sinai Health Systems Research Centre for Women's and Infants' Health BioBank (REB #18-0282-E) through a material transfer agreement for use in this biomedical research. At time of collection, the atrioventricular junction was specifically excised for use in single nuclear transcriptome studies, and ventricular myocardium was excised as a source of myocardial proteins to create 2-dimensional (2D) gels for autoantibody discovery.

Separately, developmental biology-guided differentiation protocols were used to generate fetal AV-node-like pacemaker cells (AVNLPCs) from human pluripotent cells as a source of proteins to create 2D gels for autoantibody discovery. Generally, the technique for generating chamber-specific cells is described by Zhao, Y., et al. ((2019). Cell 176 (4): 913-927 e918), the contents of which are incorporated herein by reference. The method of Zhao et al. is a scalable tissue-cultivation platform that is cell source agnostic and enables drug testing under electrical pacing. The plastic platform enabled on-line noninvasive recording of passive tension, active force, contractile dynamics, and Ca (2+) transients, as well as endpoint assessments of action potentials and conduction velocity. By combining directed cell differentiation with electrical field conditioning, electrophysiologically distinct atrial and ventricular tissues were engineered with chamber-specific drug responses and gene expression. Engineering of heteropolar cardiac tissues containing distinct atrial and ventricular ends provided spatially confined responses to serotonin and ranolazine. Uniquely, electrical conditioning for up to 8 months enabled modeling of polygenic left ventricular hypertrophy starting from patient cells.

Methods: For preparation of 2D gels, protein sources (20 week fetal ventricular myocardium, ventricular-like cardiomyocytes) were solubilized in CHAPS buffer containing 0.2% CHAPS, 20 mM leupeptin, 10 mM pepstatin A, 10 mM aprotonin, 1 mM PMSF and 5 mM DTT in 50 mM Tris, pH 7.4. The tissue suspension was sonicated for total 1 min (5 times, 20 sec each time) using a commercial sonicator (Fisher Scientific; model-60 Sonic Dismembrator) on ice and subjected to freeze-thaw twice at −80° C. followed by centrifugation at 14000 rpm for 20 min at 4° C. The tissue suspension was vortexed 5 times (total 2 min) keeping the suspension on ice in between the vortexing. The solubilized tissue was further subjected to freeze-thaw 2 times at −80° C., then centrifuged at 14000 rpm for 20 min at 4° C. The supernatant was taken out carefully without disturbing the interface. A sample of 120 micrograms of protein was placed on an isoelectric focusing strip and underwent first dimensional electropheresis according to BioRad instructions. These proteins separated by isoelectric pH were then transferred to the edge of a 2D gel (7.5% PAGE) and underwent electrophoresis in the perpendicular direction to further separate proteins based on molecular weight. These well-separated proteins on 2D gel were then exposed to maternal sera at a 1:100 dilution. Due to the scarcity of fetal and stem cell-derived proteins and complexity of gel preparation, these 2D gels were stripped and reprobed with unique sera up to three times. For western blots, commercial proteins were applied to each of 12 lanes and underwent electrophoresis. Proteins were selected based on their identification from mass spectrometry of protein target spots identified from 2D gels exposed to CHB sera, as well as their presence in both gene expression data from mouse and protein expression date from a proteomic study of human AV junctional tissue, as well as being available from a commercial source and suitable for western blot applications. These gels (all 12 lanes) were exposed to unique sera from CHB and non-CHB pregnancies.

Single-nucleus RNA sequencing (snRNA-seq) of cells dissociated from excised AV junctional tissue was performed and 20 different cell clusters were characterized including AV nodal cells based on known gene expression profiles and evaluated gene expression of autoantibody targets across these cell clusters.

Statistical Analyses: All analyses (presence of heart block outcome versus autoantibody detection) were performed with Fisher Exact test.

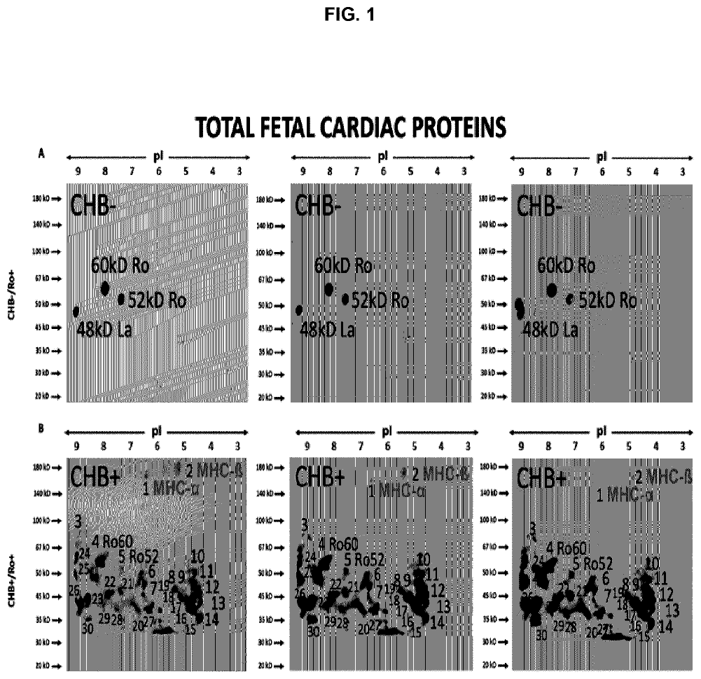

Results: Sera from three anti-Ro positive pregnancies where the fetus did not develop CHB recognized only three 2D proteins spots, corresponding to 52 kD Ro, 60 kD Ro and 48 kD La proteins (see FIG. 1, A-C). In contrast, sera from three pregnancies where the fetus developed CHB recognized at least 30 spots that were highly consistent between sera (FIG. 1, D-F).

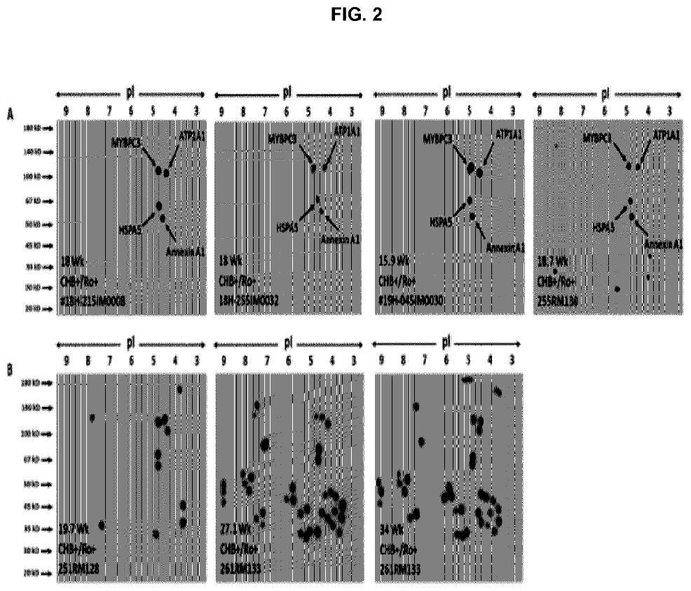

Using 2D gels of proteins derived from AVNPLCs and analyzing sera drawn sequentially from each pregnancy at 19, 24 and 34 weeks gestation, antibodies to a limited number of eight protein groups were seen at 19 weeks, expanding to additional protein groups at 24 and 34 weeks (FIG. 2). In contrast, sera from anti-Ro positive pregnancies where the fetus did not develop CHB did not react with any of these proteins (not shown). This result also indicates that AVNPLCs do not express Ro or La proteins.

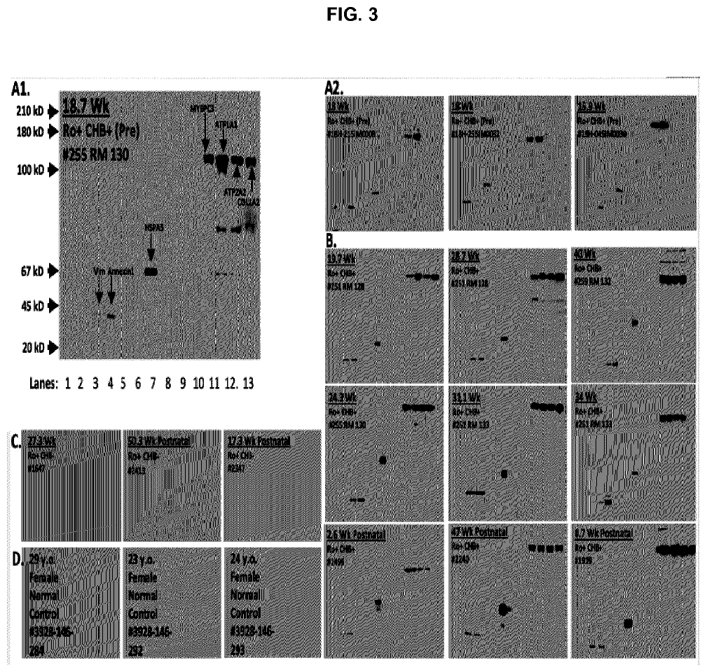

After mass spectrometry analysis of protein spots from the above studies, 12 candidate proteins available commercially were selected for Western blot studies and assessed Ro+ CHB+ sera drawn from 13 maternal samples from 7 patients (sera ranging from 18 wks. gestation through to term; FIG. 3: A1, A2 and B), 3 Ro+ CHB− sera (FIGS. 3: C) and 3 control Ro− CHB− sera from normal young women (FIG. 3: D), against western blots of all 12 proteins on one membrane (for each serum). Antibodies against seven proteins were uniquely identified from 18 wks to term in sera drawn following the onset of CHB in CHB+ pregnancies (FIG. 3: B). Antibodies to 4 of these proteins were present in 4 sera drawn prior to the onset of CHB (FIGS. 3: A1 and A2), including anti-ATP1A1 antibodies which demonstrated the strongest early signal. This protein is the isoform of Na⁺/K⁺ ATPase pump most prevalent in myocardium, and the isoform that expresses in sarcolemma but not t-tubules (which AV nodal cells do not have).

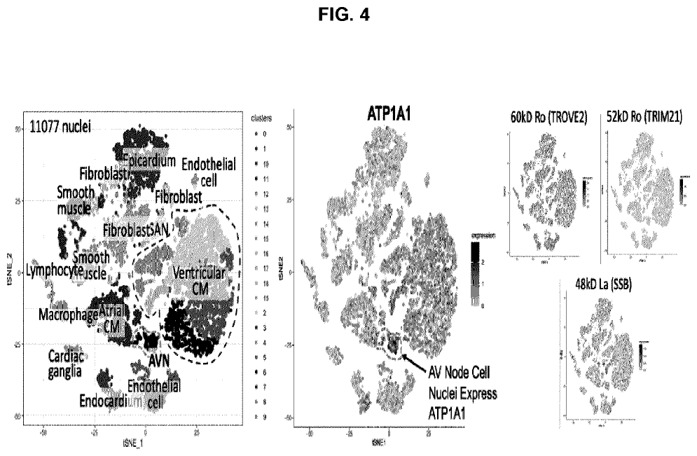

Based on single nucleus transcriptomics of cells from AV junctional tissue dissected from a 20-week gestation normal fetal heart, results were projected onto a two-dimensional t-distributed stochastic neighbour embedding (tSNE) plot. Twenty distinct cell clusters were identified, including an AV nodal cluster based on known expressed genes (FIG. 4 eft panel) ATP1A1, an identified target of CHB-related autoantibodies, was markedly overexpressed in these AV nodal cells (FIG. 4 center panel).

Conclusions: Using broad autoantibody discovery techniques, biased only toward proteins present in fetal ventricular myocardium and stem-cell derived AVNPLCs, novel CHB-associated autoantibodies were identified, including those targeting the Alpha-1 Sodium-Potassium ATPase. This subunit is highly expressed in fetal AV nodal cells from a 20-week gestation normal fetal heart, compared to other cardiac cell types.

### Example 2

In these studies, maternal sera in a discovery cohort came from anti-Ro positive pregnancies with gestational ages ranging from 15.9 to 42.6 weeks. All were participating in research ethics board approved protocols at the Hospital for Sick Children (#1000034004, R150717025, #1000029263). Maternal sera in a validation cohort came from anti-Ro positive pregnancies with gestational ages ranging from 7 to 39 weeks. All were participating in institutional review board approved studies of the Audit Committee of the University-Hospital of Padua (#6894). For the validation cohort only, the participants form a consecutive series from the outpatient clinics of University Hospital of Padua.

### Confirmation of Targets With Commercial Proteins and Antibodies

Twelve protein targets identified by mass spectrometry, confirmed to be expressed at the gene level in murine fetal atrioventricular cells and/or at the protein level in human atrioventricular junctional tissue, as described in Example 1, were used to confirm autoantibody protein targets in samples. Maternal antibodies in the discovery cohort were further confirmed by exposing multilane western blots of commercial proteins for these 12 targets to sera from heart block and non-heart block pregnancies. For four protein targets of congenital heart block maternal sera, drawn prior to the onset of heart block in the fetus, a 2D gel was also probed with individual commercial proteins to these targets to confirm the location and identity of each protein on the gel.

### Statistical Analysis

The discovery cohort consisted of archived serum samples from anti-Ro positive pregnancies and were analyzed prospectively in a case/control design using Fisher's exact test. Identified autoantibodies were assessed as diagnostic biomarkers (ability to identify the presence of congenital heart block outcome) and as predictive biomarkers (ability of biomarker to predict the subsequent development of congenital heart block). External validation was performed on an independent set of archived serum samples from a different institution and region (the University-Hospital of Padua), again analyzed prospectively in a case/control design using Fisher's exact test.

### Results

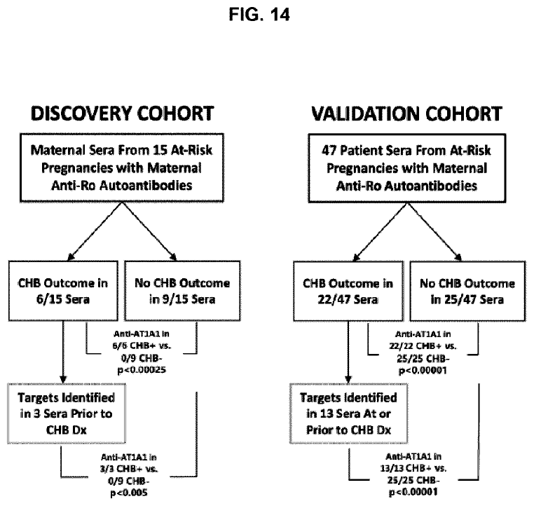

For discovery of the maternal autoantibodies associated with congenital heart block, we assessed maternal sera drawn at times ranging from 15.9 weeks gestation through to the postnatal period from 6 anti-Ro positive mothers of children with congenital heart block. Three of these were drawn preceding the detection of congenital heart block in the fetus around 18 weeks of gestation. For comparison, maternal sera drawn at times ranging from 27.3 weeks gestation through to the postnatal period from 9 anti-Ro positive women with normal children were assessed. A validation cohort consisting of 22 affected and 25 unaffected pregnancies from the University of Padua was also assessed. The sample workflow and comparisons are shown in FIG. 14.

Confirmation of maternal autoantibody targets in congenital heart block was performed by exposing the sera from the discovery cohort to multilane western blots of 12 commercial proteins identified in the bioinformatic analysis. The proteins identified were CO3 (encoded by C3 gene), VIME (VIM), ANXA1, DESP (DSP), BIP (HSPA5), ACTA (ACTA2), COF1 (CFL1), MYPC3 (MYBPC3), AT1A1 (ATP1A1), AT2A2 (ATP2A2), and CO1A2 (COL1A2). Autoantibodies to four proteins were consistently identified in anti-Ro positive sera from six pregnancies resulting in congenital heart block and were absent in sera from nine pregnancies that did not result in heart block (p<0.0005). Notably, these antibodies were present in the three pregnancies sampled prior to the development of heart block in the fetus, compared to the nine pregnancies without heart block (p<0.005).

For these four protein targets identified by early gestation maternal sera prior to the onset of congenital heart block, we confirmed the protein identities by probing additional membranes of 2D gels of ventricular-like cardiomyocyte proteins with commercial autoantibodies. This confirmed that the targets of early maternal sera in pregnancies with congenital heart block outcomes are AT1A1, MYPC3, ANXA1 and BIP.

For the validation cohort, 2D gels of ventricular-like cardiomyocyte proteins were exposed to maternal sera from 22 anti-Ro positive pregnancies with a congenital heart block outcome and compared to sera from 25 anti-Ro positive pregnancies of normal children. Autoantibodies to AT1A1 were present from 7 weeks gestation and onward in all maternal sera from 22 pregnancies resulting in congenital heart block and absent in all 25 pregnancies resulting in a normal child (p<0.00001, Fisher exact test). Beginning at 17 weeks and onward, antibodies to MYPC3, ANXA1 and BIP were also present in maternal sera from 21 pregnancies resulting in congenital heart block and absent in all 25 pregnancies resulting in a normal child (p<0.00001, Fisher exact test). In the control samples from anti-Ro positive mothers of normal children, anti-Ro antibodies were frequently but not uniformly identified using these 2D gels of ventricular-like cardiomyocyte proteins.

### Example 3

A study to determine antigenic regions of AT1A1 was conducted.

Briefly, from the whole protein sequence of AT1A1 protein, peptides of 20 amino acid lengths were synthesized (by Thermo peptide synthesis facility) starting from the N-terminus with 5 amino acid overlaps. The peptides were spotted on a nitrocellulose membrane, blocked with Pierce non-protein blocking solution, incubated with CHB+ and CHB− sera followed by incubation with anti-human IgG-HRP. After washings, bound antibodies were visualised electrochemiluminescence (ECL). The spots detected by CHB+ sera were identified.

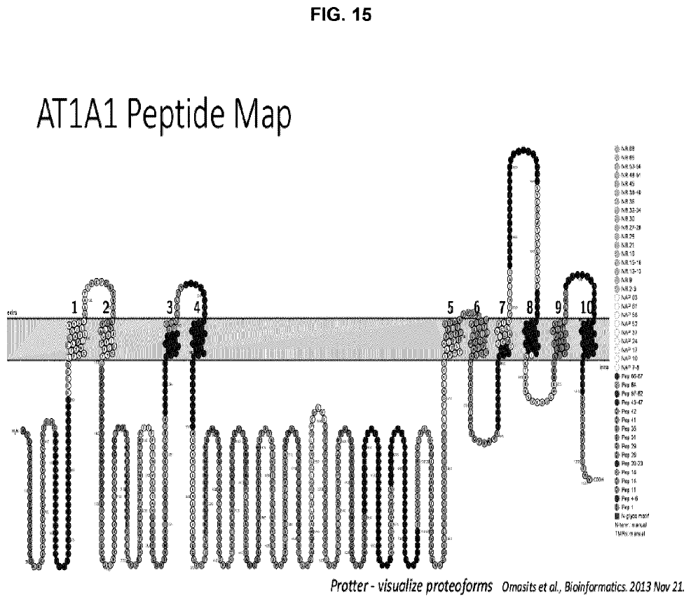

Results of the study are shown in FIG. 15. Peptides of AT1A1 comprising the following ATA1 residues were found to exhibit binding to serum CHB+ autoantibodies: residues 1-20, 151-170, 196-215, 256-275, 376-395, 421-440, 451-470, 511-530, 601-615 and 951-965 of AT1A1.

1. A method of diagnosing congenital heart block or risk of congenital heart block (CHB) in a fetal or infant subject comprising the steps of:
i) contacting a biological sample obtained from the mother of the subject with one or more target cardiomyocyte proteins or antigenic fragment(s) thereof;
ii) detecting one or more maternal autoantibodies from the biological sample which bind to at least one of the target cardiomyocyte proteins or antigenic fragments; and
iii) diagnosing the subject with congenital heart block or risk of congenital heart block when the sample contains one or more maternal autoantibodies that bind to at least one of the target cardiomyocyte proteins or antigenic fragments thereof.

2. The method of claim 1, wherein the target cardiomyocyte protein is a protein from the ventricular myocardium.

3. The method of claim 1, wherein the target cardiomyocyte protein is a protein from atrioventricular-node-like pacemaker cells.

4. The method of claim 1, wherein the target cardiomyocyte protein is selected from one or more of AT1A1, MYBPC3, AT2A2, CO1A2, HSPA5, Annexin1 and Vimentin, or antigenic fragment thereof.

5. The method of claim 4, wherein the target cardiomyocyte protein is selected from one or more of AT1A1, MYBPC3, HSPA5 and Annexin1, or antigenic fragment thereof.

6. The method of claim 1, wherein the target cardiomyocyte protein is AT1A1, or antigenic fragment thereof.

7. The method of claim 1, wherein the subject is a fetus.

8. The method of claim 1, wherein the subject is a fetus of at least 12 weeks.

9. The method of claim 1, wherein the subject is a fetus of at least 16 weeks to a newborn of up to 12 months of age.

10. The method of claim 1, wherein the subject is a fetus of at least 18 weeks.

11. The method of claim 1, wherein the biological sample is obtained prior to onset of CHB in the subject.

12. The method of claim 1, wherein the mother of the subject has or is at risk for Sjögren's syndrome, and/or has had a previous child with CHB.

13. The method of claim 1, wherein the biological sample is blood, serum, plasma, urine, saliva, cerebrospinal fluid or amniotic fluid.

14. The method of claim 1, wherein following diagnosis of the fetal subject with congenital heart block or risk of congenital heart block, the method comprises the additional step of administering hydroxychloroquine to the mother.

15. The method of claim 1, wherein following diagnosis of the fetal subject with congenital heart block, the method comprises the additional step of administering fluorinated steroids or chronotropic agents to the mother.

16. The method of claim 1, wherein following diagnosis of the fetal subject with congenital heart block, the method comprises the additional step of administering adrenocorticosteroids or sympathomimetics to the mother.

17. A kit useful for the diagnosis of CHB, said kit comprising AT1A1, MYBPC3, HSPA5 and annexin1 antigens or antigenic fragments thereof, and optionally one or more antigens selected from AT2A2, CO1A2 and vimentin or antigenic fragments thereof, immobilized on a solid support.

18. The kit of claim 17, wherein the solid support is selected from a nitrocellulose, polyvinylidene difluoride (PVDF), or cationic nylon membrane, or microparticles.

19. The kit of claim 17 or claim 18, wherein one of said antigens is an AT1A1 peptide comprising amino acid residues 1-20, 151-170, 196-215, 256-275, 376-395, 421-440, 451-470, 511-530, 601-615 and/or residues 951-965 of AT1A1.

20. A method comprising the steps of:
i) contacting a biological sample obtained from the mother of a fetal or infant subject with one or more fetal or infant cardiomyocyte proteins found in the ventricular myocardium, and/or one or more antigenic fragments of said cardiomyocyte proteins; and
ii) detecting one or more maternal autoantibodies from the biological sample which bind to at least one of the cardiomyocyte proteins or antigenic fragments.

21. The method of claim 20, wherein the cardiomyocyte protein is selected from one or more of AT1A1, MYBPC3, AT2A2, CO1A2, HSPA5, Annexin1 and Vimentin, or an antigenic fragment thereof.

22. The method of claim 21, wherein the target cardiomyocyte protein is selected from one or more of AT1A1, MYBPC3, HSPA5 and Annexin1, or antigenic fragment thereof.

23. The method of claim 20, wherein the biological sample is blood, serum, plasma, urine, saliva, cerebrospinal fluid or amniotic fluid.

24. The method of claim 23, wherein the biological sample blood, serum or plasma.

25. The method of any one of claims 20 to 24, wherein the biological sample is obtained prior to onset of CHB in the subject.

26. An antigenic fragment of AT1A1 comprising amino acid residues 1-20, 151-170, 196-215, 256-275, 376-395, 421-440, 451-470, 511-530, 601-615 and/or residues 951-965 of AT1A1.
